(19) United States
(12) Patent Application Publication
(10) Pub. No.: US 2023/0022892 A1
(43) Pub. Date: Jan. 26, 2023

(54) BREATHING TUBE FOR USE WITH SPIROMETERS THAT EMPLOY ULTRASONIC MEASUREMENT SYSTEMS

(71) Applicant: SDI Diagnostics, Inc., Easton, MA (US)

(72) Inventor: Michael BOYLE, Easton, MA (US)

(21) Appl. No.: 17/864,013

(22) Filed: Jul. 13, 2022

Related U.S. Application Data
(60) Provisional application No. 63/221,793, filed on Jul. 14, 2021.
(60) Provisional application No. 63/301,921, filed on Jan. 21, 2022.

(51) Int. Cl. A61M 16/04; A61B 5/08

(52) U.S. Cl. CPC A61M 16/0431; A61B 5/082; A61M 16/0418

(57) ABSTRACT

Breathing tubes for use with a spirometer. The breathing tubes include a body having an oval interior cross-sectional shape and are configured to engage interior surfaces of the spirometer, so as to ensure the proper orientation of the body within the spirometer.

### CROSS REFERENCE TO RELATED APPLICATIONS

This application claims the benefit of priority to U.S. Provisional Patent Application No. 63/221,793, filed Jul. 14, 2021, and U.S. Provisional Patent Application No. 63/301,921, filed Jan. 21, 2022, the disclosures of which are incorporated by reference herein in their entireties.

### FIELD OF THE INVENTION

Embodiments of the present invention relate to breathing tubes, and more particularly, breathing tubes for use with ultrasonic spirometers.

### BACKGROUND OF THE INVENTION

A spirometer is an apparatus for measuring the volume of air inspired and expired by the lungs of a patient. A spirometer measures ventilation, i.e., the movement of air into and out of the lungs. The spirogram identifies two different types of abnormal ventilation patterns: obstructive and restrictive. There are various types of spirometers that use several different methods for measurement (e.g., pressure transducers, ultrasonic, water gauge).

A spirometer is the main piece of equipment used in conducting basic Pulmonary Function Tests (PFTs) on patients. Lung diseases such as asthma, bronchitis, and emphysema may be ruled out from the results of the PFTs. In addition, a spirometer often is used for finding the cause of shortness of breath, assessing the effect of contaminants on lung function, the effect of medication, and evaluating progress for disease treatment.

In operation, a medical professional instructs a test subject/patient to exhale forcefully into the breathing tube. The expired gases are directed across the ultrasonic transmitter/receiver system in the spirometer. This system is designed to measure the flow by transit times with the flow and against the flow. By forcefully blowing into the breathing tube there exists a possibility of expelling harmful pathogens into the test area environment. Breathing tubes known in the art have no mechanism to prevent this potentially very dangerous occurrence.

Spirometers use disposable mouthpieces to eliminate cross-contamination of patients. Many of the mouthpieces contain viral/bacterial filters that offer an extra layer of protection for the patient and the test administrator. Spirometry involves exhaling forcefully into the device. Unless a filter is used, any viruses or bacteria from the patient are blown into the test environment. This can be very unsafe for the medical professional(s) treating the patient and any others in the surrounding area.

One model of spirometer is made in Switzerland by Ndd Medizintechnik, AG. This spirometer uses a “breathing tube” as the mouthpiece and is disclosed in U.S. Pat. No. 10,786,178. The shape of the breathing tube disclosed in the '178 Patent is intended to only allow correct insertion of the breathing tube into the spirometer. This is accomplished by utilizing a “polygonal transversal cross section along the longitudinal axis of the breathing tube” which can only be used in the correct orientation. The breathing tube of the '178 Patent also has at least a pair of beveled outer sides opposing on another and extending longitudinal axis.

### BRIEF SUMMARY OF THE INVENTION

Embodiments of the present invention provide breathing tubes for use with a spirometer. The breathing tubes include a body having an oval interior cross-sectional shape and are configured to engage interior surfaces of the spirometer, so as to ensure the proper orientation of the body within the spirometer. The breathing tubes also include a mouthpiece connected to the body.

In other embodiments of the present invention, the breathing tubes include a body having an oval interior cross-sectional shape and including one or more protrusions that are configured to engage the interior surfaces of the spirometer, so as to ensure the proper orientation of the body within the spirometer. The one or more protrusions are arranged so that the body can only be inserted into the spirometer in the correct orientation. The breathing tubes also include a mouthpiece connected to the body.

Other embodiments are also described and recited herein.

### BRIEF DESCRIPTION OF THE DRAWINGS

For the purpose of illustration, certain embodiments of the present invention are shown in the drawings described below. Like numerals in the drawings indicate like elements throughout. It should be understood, however, that the invention is not limited to the precise arrangements, dimensions, and instruments shown. In the drawings:

FIG. 1a is a front elevational view of a breathing tube according to an embodiment (first) of the present invention;

FIG. 1b is a side elevational view of the breathing tube of FIG. 1a;

FIG. 1c is a top perspective view of the breathing tube of FIG. 1a;

FIGS. 2a and 2b are cross-sectional views of the breathing tube of FIG. 1a in use with a spirometer;

FIG. 3a is an enlarged partial perspective view of the breathing tube of FIG. 1a

FIG. 3b is an enlarged partial interior view of the breathing tube of FIG. 1a;

FIG. 4a shows enlarged front and side views of portions of the breathing tube of FIG. 1a;

FIG. 4b shows enlarged cross-sectional views of portions of the breathing tube of FIG. 1a;

FIG. 5 is a top perspective view of a breathing tube according to another (second) embodiment of the present invention;

FIGS. 6a and 6b are cross-sectional views of the breathing tube of FIG. 5 in use with a spirometer;

FIG. 7 is a top perspective view of a breathing tube with an integral filter according to another (third) embodiment of the present invention; and

FIG. 8 is a cross-sectional view of the breathing tube of FIG. 7 showing the integral filter therein.

### DETAILED DESCRIPTION OF THE INVENTION

It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

### Breathing Tubes

Disclosed herein are breathing tubes for use with a spirometer.

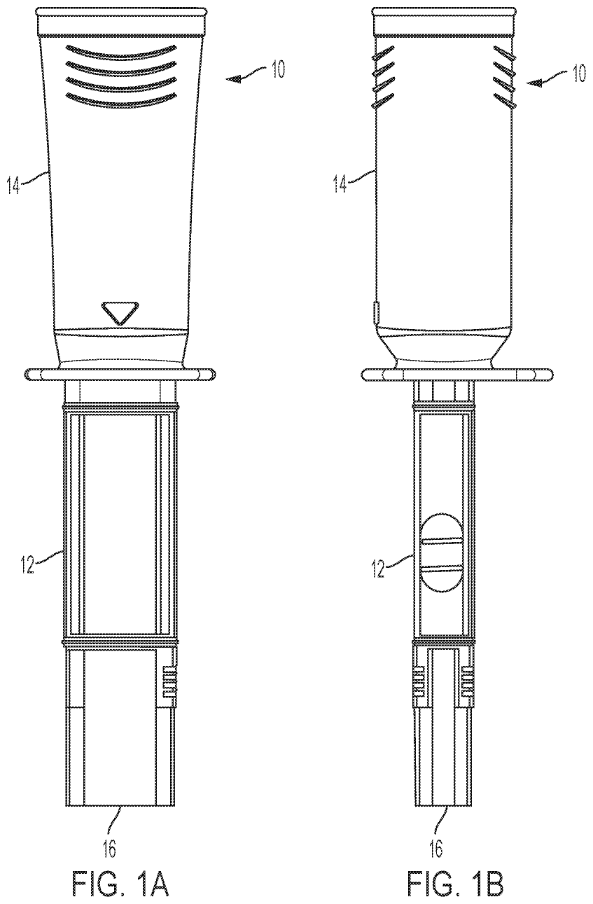

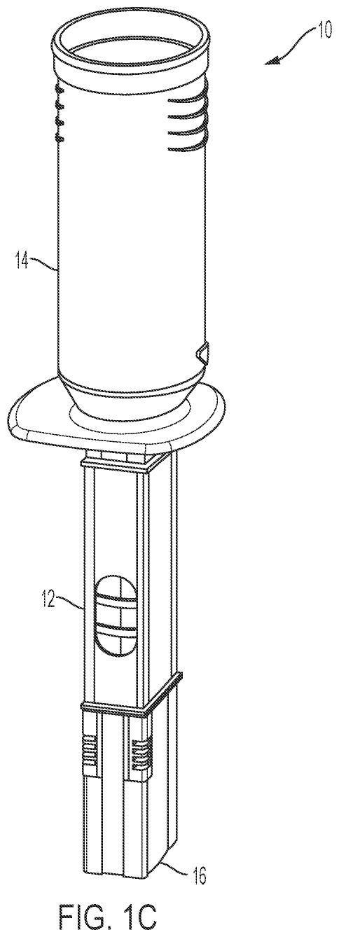

An embodiment of the breathing tube of the present invention is illustrated in FIGS. 1a, 1b and 1c. The breathing tube 10 includes a body 12 and a mouthpiece 14 connected to the body 12 opposite an end 16 of the body 12. In this and other embodiments, the mouthpiece 14 of the breathing tube 10 does not include any integral filter.

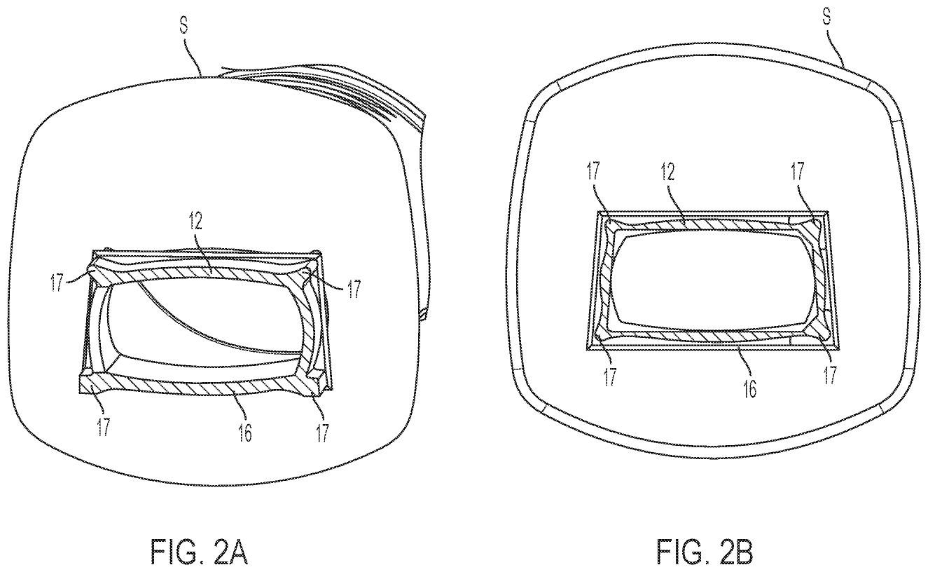

FIGS. 2a and 2b are cross-sectional views of the breathing tube 10 in use with a spirometer S. Specifically, the end 16 of the body 12 of the breathing tube 10 opposite the mouthpiece 14 is shown as having been inserted into an opening in the spirometer S. The body 12 has a modified oval cross-sectional shape, in contrast to bodies of prior art breathing tubes that have a trapezoidal shape with straight sides.

The body 12 also includes four protrusions 17 formed on four respective end corners of the body 12 that are configured to engage the interior surfaces of the spirometer S, so as to ensure the proper orientation of the body 12 of the breathing tube 10 within the spirometer S. In other words, the protrusions 17 are arranged so that the body 12 of the breathing tube 10 can only be inserted into the spirometer S in the correct orientation. In various alternate embodiments, the body 12 has fewer or more than four protrusions 17 (e.g., 1, 2, 3, 5, 6 or 8 protrusions), and the protrusions 17 may be formed at other (i.e., non-corner) locations on the body 12.

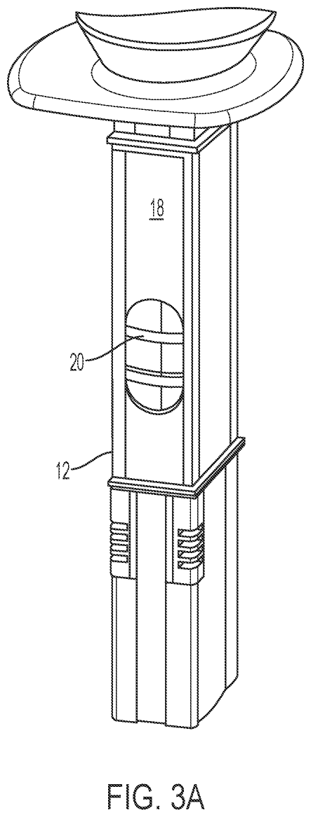

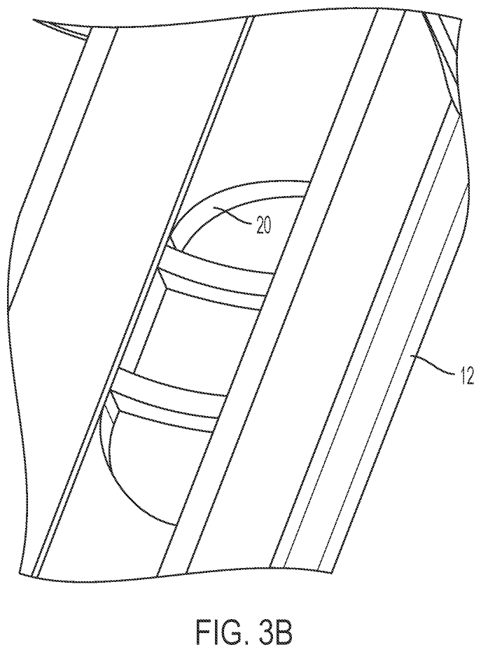

FIGS. 3a and 3b further illustrate the body 12 of the breathing tube 10, which includes a front surface 18 that is rounded/curved, and therefore not a flat surface. The curved front surface 18 imbues the body 12 with an oval cross section, thereby maximizing the internal dimensions to permit greater air flow through the body 12.

In various embodiments, the curved front surface 18 includes one or more windows 20 formed therein. The window(s) 20 is/are configured to allow the ultrasound/ultrasonic energy to be transmitted between transmitting and receiving components of a measuring system of the spirometer.

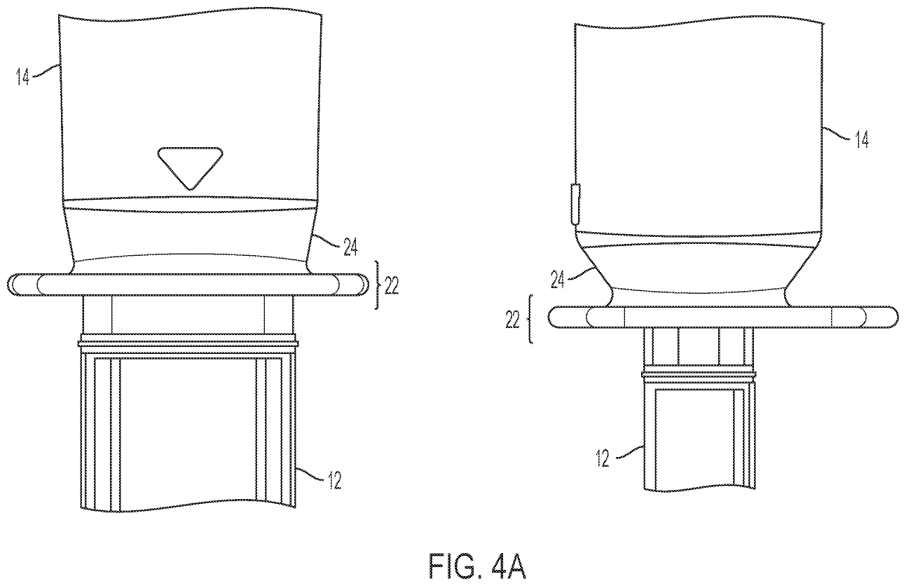

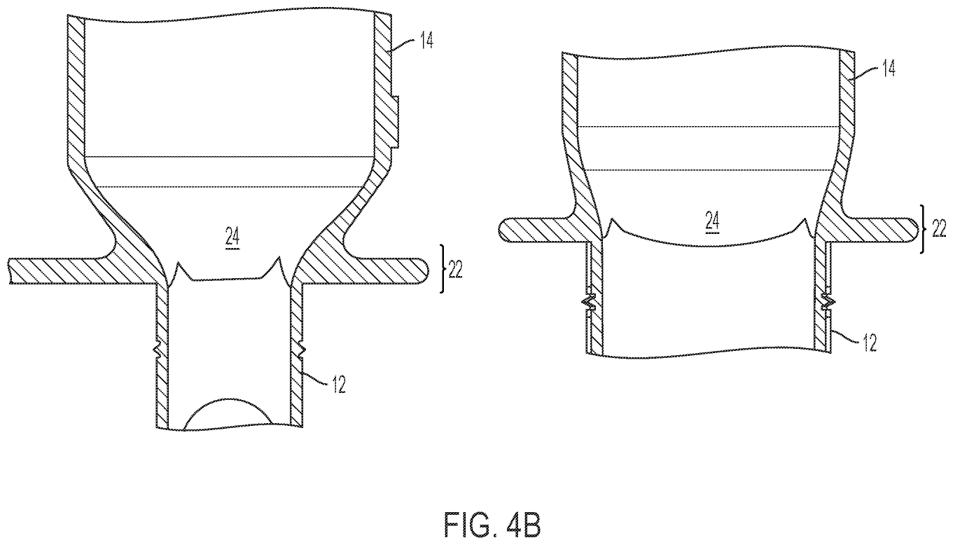

FIGS. 4a and 4b illustrate the interface 22 between the body 12 and the mouthpiece 14 of the breathing tube 10. In various embodiments, the mouthpiece 14 includes an enlarged throat area 24 proximate the interface 22.

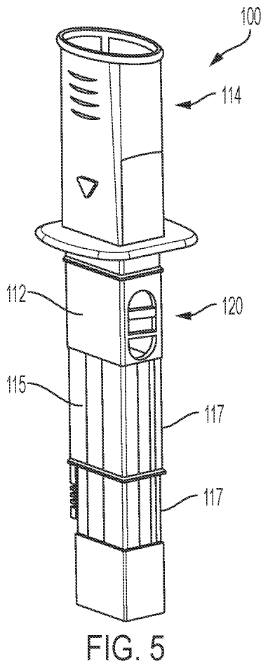

A second embodiment of the breathing tube of the present invention is illustrated in FIG. 5. The breathing tube 100 includes a body 112 having an exterior surface 110 that is at least partially flat, as well as an interior surface 115 that is at least partially curved, and a mouthpiece 114 connected to the body.

In various embodiments, the exterior surface 110 of the body 112 includes one or more windows 120 formed therein. The window(s) 120 is/are configured to allow the ultrasound/ultrasonic energy to be transmitted between the transmitting and receiving components of the measuring system of the spirometer.

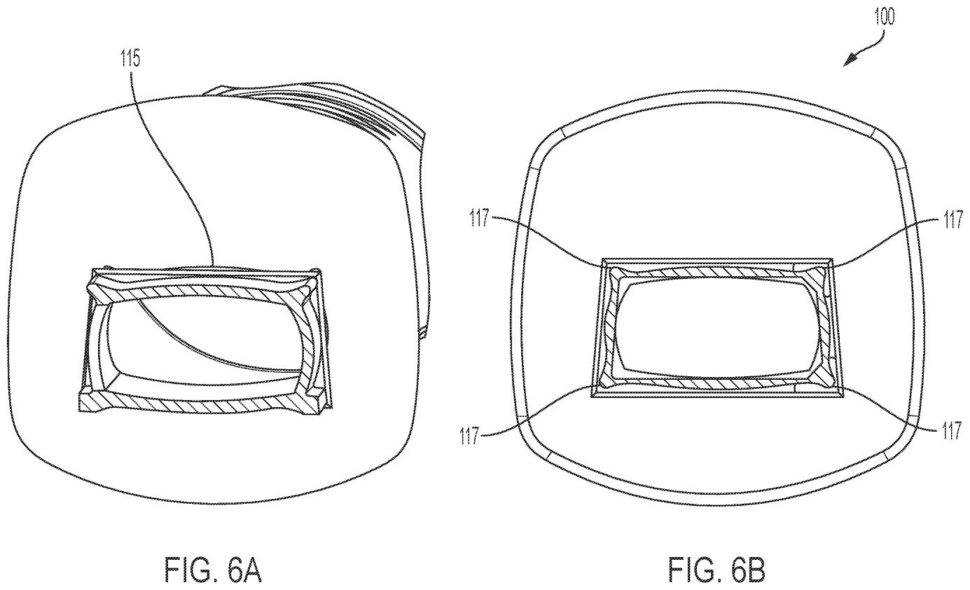

FIGS. 6a and 6b are cross-sectional views of the breathing tube 100 in use with a spirometer S. The body 112 of the breathing tube 100 has an oval internal cross-sectional shape (i.e., curved interior surface 115), in contrast to bodies of prior art breathing tubes that have a trapezoidal shape with straight sides.

The body 112 of the breathing tube 100 also has four protrusions 117 formed on four respective end corners thereof that are configured to engage the interior surfaces of the spirometer S, so as to ensure the proper orientation/location of the body of the breathing tube 100 within the spirometer S. In other words, the protrusions 17 are arranged so that the body 112 of the breathing tube 100 can only be inserted into the spirometer S in the correct orientation. In various alternate embodiments, the body 112 has fewer or more than four protrusions 117 (e.g., 1, 2, 3, 5, 6 or 8 protrusions), and the protrusions 117 may be formed at other (i.e., non-corner) locations on the body 112.

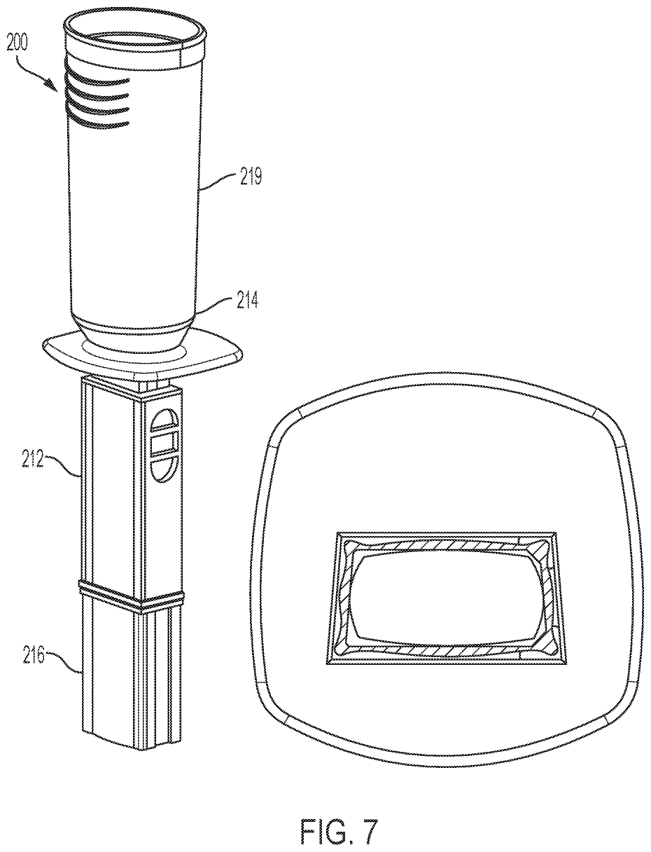

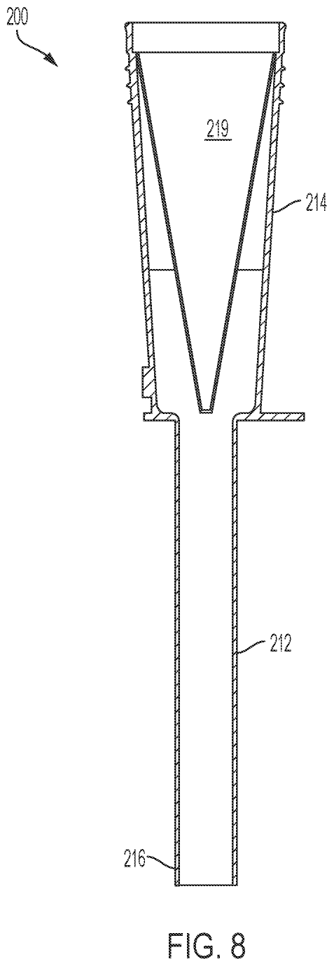

A third embodiment of the breathing tube of the present invention is illustrated in FIGS. 7 and 8. The breathing tube includes a body 212 having a mouthpiece 214 and opposite end 216. The mouthpiece 214 of the breathing tube 200 of this embodiment includes a filter 219. In various embodiments, the filter 219 is built-in/integral to the mouthpiece 214. The filter is an anti-viral/ anti-bacterial filter. In various embodiments, the mouthpiece 214 is enlarged to incorporate/accommodate the filter 219 therein.

In various embodiments, the filter 219 includes high-efficiency filter media to capture all, or most, of the harmful pathogens that may be expelled by a test subject/patient during a test so that the medical professional/test administrator and others are protected from infection. Each test subject/patient uses a different disposable breathing tube to avoid cross contamination from any possible infectious agents.

In various embodiments, the filter 219 is an electrostatic media made from polypropylene that has a permanent charge, which is the standard media typically used for anti-viral/bacterial filters for pulmonary testing use. One example of this polypropylene is Technostat® polypropylene (Superior Felt & Filtration, LLC, McHenry, Ill.)

Other than the inclusion of the filter 219 therein, the breathing tube 200 is generally the same, structurally and functionally, as the breathing tubes 10, 100 of the first and second embodiments disclosed above.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, examples, etc., described herein and as such can vary. The terminology used herein is for the purpose of describing particular embodiments only, and is not intended to limit the scope of the present invention, which is defined solely by the claims.

Other terms are defined herein within the description of the various aspects of the invention.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A breathing tube for use with a spirometer, the breathing tube comprising:
a body having an oval interior cross-sectional shape and being configured to engage interior surfaces of the spirometer, so as to ensure the proper orientation of the body within the spirometer; and
a mouthpiece connected to the body.

2. The breathing tube of claim 1, wherein the body includes one or more protrusions that are configured to engage the interior surfaces of the spirometer.

3. The breathing tube of claim 2, wherein the one or more protrusions includes four protrusions.

4. The breathing tube of claim 3, wherein the four protrusions are formed on four respective end corners of the body.

5. The breathing tube of claim 1, wherein the one or more protrusions are arranged so that the body can only be inserted into the spirometer in the correct orientation.

6. The breathing tube of claim 1, wherein the body includes a curved front surface.

7. The breathing tube of claim 6, wherein the curved front surface includes one or more windows formed therein, and wherein the one or more windows are configured to allow ultrasound/ultrasonic energy to be transmitted between transmitting and receiving components of a measuring system of the spirometer.

8. The breathing tube of claim 1, wherein the body includes an interior surface that is at least partially curved.

9. The breathing tube of claim 1, wherein the body includes an exterior surface that is at least partially flat.

10. The breathing tube of claim 9, wherein the exterior surface includes one or more windows formed therein, and wherein the one or more windows are configured to allow ultrasound/ultrasonic energy to be transmitted between the transmitting and receiving components of the measuring system of the spirometer.

11. The breathing tube of claim 1, wherein the mouthpiece includes a filter therein.

12. The breathing tube of claim 11, wherein the mouthpiece is enlarged to accommodate the filter therein.

13. The breathing tube of claim 11, wherein the filter is an integral filter.

14. The breathing tube of claim 11, wherein the filter is an anti-viral/anti-bacterial filter.

15. The breathing tube of claim 11, wherein, the filter includes an electrostatic media made from polypropylene that has a permanent charge.

16. A breathing tube for use with a spirometer, the breathing tube comprising:
a body having an oval interior cross-sectional shape and including one or more protrusions that are configured to engage the interior surfaces of the spirometer, so as to ensure the proper orientation of the body within the spirometer, wherein the one or more protrusions are arranged so that the body can only be inserted into the spirometer in the correct orientation; and
a mouthpiece connected to the body.

17. The breathing tube of claim 16, wherein the one or more protrusions includes four protrusions that are formed on four respective end corners of the body.

18. The breathing tube of claim 16, wherein a surface of the body includes one or more windows formed therein, and wherein the one or more windows are configured to allow ultrasound/ultrasonic energy to be transmitted between the transmitting and receiving components of the measuring system of the spirometer.

19. The breathing tube of claim 16, wherein the mouthpiece includes an anti-viral/anti-bacterial filter therein.

20. The breathing tube of claim 19, wherein the filter is an integral filter.
